(19) United States
(12) Patent Application Publication
(10) Pub. No.: US 2021/0030671 A1
(43) Pub. Date: Feb. 4, 2021

(54) LIQUID DOSAGE FORMS OF CINACALCET OR SALT THEREOF

(71) Applicant: FTF PHARMA PRIVATE LIMITED, Sanand, Ahmedabad (IN)

(72) Inventors: Swati NAGAR, Amreli (IN); Sandip MEHTA, Ahmedabad (IN); Manish UMRETHIA, Ahmedabad (IN); Jayanta Kumar MANDAL, Ahmedabad (IN)

(21) Appl. No.: 17/043,988

(22) PCT Filed: Mar. 30, 2019
(86) PCT No.: PCT/IB2019/052643

§ 371 Date: Sep. 30, 2020

(30) Foreign Application Priority Data
Mar. 30, 2018 (IN) 201821012059

(51) Int. Cl. A61K 9/00; A61K 9/10; A61K 31/137

(52) U.S. Cl. CPC A61K 9/0095; A61K 31/137; A61K 9/10

(57) ABSTRACT

Cinacalcet is approved and marketed as hydrochloride salt in a solid dosage form indicated for the treatment of secondary hyperparathyroidism resulting from chronic kidney disease and for the treatment of hypercalcemia in patients with either parathyroid carcinoma or hyperparathyroidism. Current marketed products are not allowed to be divided and to be taken whole during administration. Patients having swallowing difficulty may not show adherence to such regimen. The present invention therefore provides liquid dosage forms of Cinacalcet or pharmaceutically acceptable salt thereof.

### FIELD OF THE INVENTION

The present invention relates, in general to the pharmaceutical field, and more precisely it relates to the liquid dosage forms comprising calcimimetic agent that increases the sensitivity of the calcium-sensing receptor to activation by extracellular calcium viz. Cinacalcet or pharmaceutically acceptable salt thereof. In particular, the present invention relates to ready to use, liquid dosage forms comprising Cinacalcet or pharmaceutically acceptable salt thereof. The liquid dosage forms of the present invention are useful for the treatment of at east one disease chosen from hyperparathyroidism, such as primary hyperparathyroidism and secondary hyperparathyroidism, hyperphosphonia, hypercalcenia, and elevated calcium-phosphorus product.

### BACKGROUND OF THE INVENTION

Cinacalcet is chemically described as N-[1-(R)-(−)-(1-naphthyl)ethyl]-3-[3-(trifluoromethyl)phenyl]-1-aminopropane and has the following structural formula:

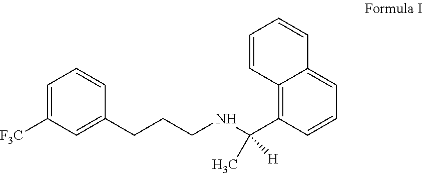 [SMILES: C[C@@H](NCCCc1cccc(C(F)(F)F)c1)c1cccc2ccccc12]

Empirical formula of Cinacalcet free base is C₂₂H₂₂F₃N with a molecular weight of 357.4 gm/mol. It has one chiral centre having an (R)-absolute configuration. The (R)-enantiomer is the more potent enantiomer and has been shown to be responsible for pharmacodynamic activity. Cinacalcet is commercially available in particular as hydrochloride salt. Cinacalcet hydrochloride is a white to off-white, crystalline solid which is soluble in methanol or 95% ethanol and slightly soluble in water.

Prior art reveals that Cinacalcet or pharmaceutically acceptable salt thereof has been formulated into solid dosage forms. Liquid dosage forms of Cinacalcet or pharmaceutically acceptable salt thereof are not much explored by the formulation scientists. US 20170312223 discloses sprinkle compositions of Cinacalcet in the form of capsule dosage forms comprising coated cores. EP 3116487 discloses tablet compositions of Cinacalcet or pharmaceutically acceptable salt thereof wherein the composition is free from binding agent and prepared by wet granulation. US 20160143863 discloses disintegrant free tablet or capsule compositions of Cinacalcet.

Because of Cinacalcet's relatively low solubility (<1 μg/mL in water), cinacalcet suffers from a sub-linear increase in absorption with increasing dose and increased absorption when administered with meals (Clin Pharmacokinet; 2009, 48 (5), 303-311). Specifically, it has been found that when Sensipar® is co-administered with a high fat meal in healthy subjects, cinacalcet Cmax and AUC^{∞} values increase by 82% and 68%, respectively, when compared to fasted state values. Also, when administered with a low fat meal, Cmax and AUC^{∞} values increased by 65% and 50%, respectively.

These findings confirm that food will significantly affect cinacalcet absorption, and that meal composition will introduce variability in this level of absorption. As cinacalcet shows a positive food effect (as described above), the product label states that Sensipar® should be taken with food or shortly after a meal, to achieve a maximal level of drug absorption using this tablet dosage form (Sensipar® (cinacalcet) tablets: US prescribing information, URL: http://pi.amgen.com/united_states/sensipar/sensipar_pi_hcp_english.pdf). Requiring patients to administer their daily medication with meals to boost drug absorption does however introduce compliance issues, particularly if patients are also taking other medications that, for example, require administration on an empty stomach. In addition, there is also the fact that the type of meal will also affect how much cinacalcet is absorbed, creating an additional source of variability. WO 2016066611 therefore discloses pharmaceutical compositions comprising Cinacalcet, a lipid component or either a medium-chain glyceride (MCG) or long-chain glyceride (LCG), or a propylene glycol fatty acid ester, or a suitable blend of these lipid components, and a non-ionic surfactant having a HLB of at least 6.

U.S. Pat. No. 9,012,511 & US 20150216822 discloses stable nanoparticulate Cinacalcet composition comprising (a) solid particles of Cinacalcet or a pharmaceutically acceptable salt thereof having an effective average particle size of less than about 2000 nm; and (b) at least one surface stabilizer.

There are other prior arts also which disclose solid pharmaceutical compositions of Cinacalcet or pharmaceutically acceptable salt thereof such as tablets or capsules, e.g. EP 1663182 discloses rapid dissolution formulation of Cinacalcet hydrochloride. EP 2314286 & EP 2334284 discloses tablet compositions of Cinacalcet or pharmaceutically acceptable salt thereof. EP 2490674 discloses immediate release tablet compositions prepared through melt-granulation process. EP 2730279 discloses immediate release tablet formulations of Cinacalcet. WO 2014096277 discloses tablet composition comprising Cinacalcet hydrochloride. EP 2821067 discloses rapid dissolution formulation of Cinacalcet. WO 2015150944 discloses solid oral pharmaceutical composition comprising Cinacalcet or a salt thereof devoid of any disintegrating agent. EP 2934485 discloses tablet composition comprising Cinacalcet hydrochloride. US 20150306049 discloses immediate release tablet formulations of Cinacalcet. US 20150328172 discloses tablet composition comprising Cinacalcet hydrochloride.

Cinacalcet is a calcium-sensing receptor agonist. It is commercially available as tablets and marketed as Sensipar® in the United States and Australia, and as Mimpara® in Europe. These tablets are formulated as light-green, film-coated, oval-shaped tablets for oral administration in the strengths of equivalent to 30 mg, 60 mg, and 90 mg free base. Each tablet contains pregelatinized starch, microcrystalline cellulose, povidone, crospovidone, colloidal silicone dioxide, and magnesium stearate. Tablets are coated with color (Opadry® II green) and clear film-coat (Opadry® clear), carnauba wax, and Opacode® black ink. Cinacalcet is indicated for the treatment of secondary hyperparathyroidism resulting from chronic kidney disease and for the treatment of hypercalcemia in patients with either parathyroid carcinoma or hyperparathyroidism. Currently, Cinacalcet is also in Phase III clinical trials in pediatric patients with secondary hyperparathyroidism (SHPT) and chronic kidney disease (CKD) on dialysis wherein Cinacalcet capsules are sprinkled onto soft food or suspended into a liquid suspension for oral administration.

Compared to the conventional tablets and capsules, oral liquid dosage forms including solutions, syrups, suspensions, elixirs, and concentrates offer unique advantages to many patients. For example, liquids may provide better patient compliance for those with swallowing difficulties and better dosage control versus a fixed tablet dose. Hence, liquid dosage forms are generally formulated for use in geriatric and pediatric patients. However, there are also a number of “challenges” surrounding the formulation and development of these forms.

Children generally reject taking medicine which does not have a favorable shape, taste, flavor, etc. However, if a child who needs to take a medicine, rejects taking it, he might never recover from his condition. When a child is unable to take medicine orally, it is intravenously administered, and he and his caregivers then may experience stress. Syrups and suspensions are considered as favorable types of dosage forms in which to orally administer medicine to infants and children. However, they may have disadvantages such as solubility, a bad taste, portability problems or required refrigerator storage. Palatability is one of the main elements of patient acceptability of an oral pediatric medicine. Palatability is defined as the overall appreciation of an oral medicinal product in relation to its smell, taste, aftertaste and feeling in the mouth. Design of the formulation of an oral pediatric medicine should be considered together with its palatability.

Sensipar® prescribing information suggests that for all indications Sensipar® tablets should always be taken whole and not divided (Sensipar® (Cinacalcet), United States: https://www.accessdata.fda.gov/drugsatfda_docs/label/2017/021688s023lbl.pdf). It is therefore desirable to have liquid dosage forms of Cinacalcet or pharmaceutically acceptable salt thereof. Liquid dosage forms offer unique advantages to many patients having swallowing difficulties such as pediatric patients and geriatric patients or other patients who are unable to take solid oral therapy and may provide better patient compliance.

### OBJECTS OF THE INVENTION

Because of their liquid character, liquid dosage forms represent an ideal dosage form for patients who have difficulty swallowing tablets or capsules. This factor is of particular importance in administration of drugs to children and aged patients. Further, as mentioned above, Sensipar® tablets should always be taken whole and not divided. It is therefore principal object of the present invention to provide liquid dosage forms of Cinacalcet or pharmaceutically acceptable salt thereof. The liquid dosage forms of the present invention are useful for administering to pediatric, geriatric patients and other patients who are unable to take solid oral therapy. The liquid dosage forms according to the present invention include liquids, liquid dispersions, suspensions, solutions, emulsions, sprays, spot-on, syrups, elixirs, drops, gels, solution-gels, concentrates and the like.

Suspensions possess certain advantages over other liquid dosage forms. Some drugs are insoluble in all acceptable media and must, therefore, be administered as a tablet, capsule, or as a suspension. In addition, disagreeable tastes can be masked by a suspension of the drug or a derivative of the drug. Drugs in suspension are chemically more stable than in solution. In another object, the present invention therefore provides suspension dosage forms of Cinacalcet or pharmaceutically acceptable salt thereof.

Liquid dosage forms are designed as ready to use liquids and as powder for reconstitution into liquid orals like syrups, solutions, suspensions and emulsions. Powder for reconstitution may require skills & expertise and needs to be prepared by a healthcare provider and may not be prepared by the patient or caregiver. The reconstitution process may also be a time consuming process and the patient cannot be benefited by the immediate dose of Cinacalcet as and when required. In such a situation, ready to use, liquid dosage forms of Cinacalcet may be very useful and the patients can be given required doses immediately using ready to use, liquid dosage forms of Cinacalcet. Therefore, a yet another object of the present invention is to provide ready to use, liquid dosage forms of Cinacalcet or pharmaceutically acceptable salt thereof.

A yet another object of the present invention is to provide ready to use, liquid dosage forms of Cinacalcet or pharmaceutically acceptable salt thereof comprising one or more pharmaceutically acceptable excipients or additives selected from the group comprising of vehicles, solvents/co-solvents, solubilizers, suspending agents/thickening agents/viscosity modifying agents, anti-foaming agents, anti-caking agents, surfactants, pH adjusting agents and/or pH modifying agents and/or buffering agents or any combination thereof. The ready to use, suspension dosage forms of the present invention may further comprise one or more agents selected from the group comprising of preservatives, sweetening agents, flavoring agents and coloring agents or any combination thereof. One or more of the above mentioned excipients or additives may be omitted depending upon the preparation of the final dosage form.

A yet another object of the present invention is to provide liquid dosage forms of Cinacalcet or pharmaceutically acceptable salt thereof having palatability, prolonged stability and comparable bioavailability when compared to the marketed drug.

A yet another object of the present invention is to provide process for the preparation of liquid dosage forms of Cinacalcet or pharmaceutically acceptable salt thereof.

A yet another object of the present invention is to provide method for the treatment of a disease or disorder that can be treated by altering a subject's calcium receptor activity. A yet another object of the present invention is to provide method for the treatment of a disease chosen from hyperparathyroidism, such as primary hyperparathyroidism and secondary hyperparathyroidism, hyperphosphonia, hypercalcemia, and elevated calcium-phosphorus product comprising administering to a patient, such as human, an effective dosage amount of a liquid dosage form comprising Cinacalcet or pharmaceutically acceptable salt thereof and one or more pharmaceutically acceptable excipients or additives as disclosed herein.

A yet another object of the present invention is to use the liquid dosage forms of Cinacalcet or pharmaceutically acceptable salt thereof prepared according to the present invention for the treatment of a disease or disorder that can be treated by altering a subject's calcium receptor activity. A yet another object of the present invention is to use the liquid dosage forms of Cinacalcet or pharmaceutically acceptable salt thereof prepared according to the present invention for the treatment of a disease chosen from hyperparathyroidism, such as primary hyperparathyroidism and secondary hyperparathyroidism, hyperphosphonia, hypercalcemia, and elevated calcium-phosphorus product.

### DETAILED DESCRIPTION OF THE INVENTION

Characteristics of an active drug are of major concern in developing an oral liquid dosage formulation. The major challenges in developing oral liquid dosage forms are (i) the stability of a drug in solution, (ii) the solubility of a drug at the required level, and (iii) an acceptable taste. It is the effective use of excipients, which allows formulators overcome these challenges. Additionally, an excipient's compatibility with a drug in the solid state cannot infer the same compatibility in solution.

The decision to develop a solution, syrup or a suspension of a drug is influenced by many factors like solubility and the desired release profile of the drug and properties of the base vehicle like surface tension, viscosity, boiling point, and specific heat of solution, all of which may be affected in various ways. In case of clear liquids, lack of solubility of the drug in the base vehicle may demand the need for miscible co-solvents. Similarly, a miscible solvent may be needed to decrease the solubility of the drug in a primary vehicle in formulating a suspension.

The therapeutic utility of drugs involves the application of dosage forms/delivery systems, which serve as carrier systems together with several excipients to deliver the active therapeutic agent to the site of action. Suspensions are an important class of pharmaceutical dosage forms that may be given by many routes, including oral, topical, parenteral, and also used in the eye for ophthalmic purposes. Surprisingly, large proportions of new drug candidates that are emerging are predominantly water insoluble and, therefore, demonstrate poor bioavailability in the solution dosage form. While suspensions present a viable formulation option for many drugs, particularly for water insoluble, hydrophobic drug substances, there are certain criteria that a well-formulated suspension should meet.

The suspension dosage form has long been used for poorly soluble active ingredients for various therapeutic indications. Development of stable suspensions over the shelf life of the drug product continues to be a challenge on many fronts. Drugs from suspension formulations typically exhibit an improved bioavailability when compared to the same drug formulated as a tablet or capsule.

A good understanding of the fundamentals of disperse systems is essential in the development of a suitable pharmaceutical suspension. The development of a suspension dosage form follows a very complicated path. The selection of the proper excipients (surfactants, viscosity imparting agents etc.) is important. The particle size distribution in the finished drug product dosage form is a critical parameter that significantly impacts the bioavailability and pharmacokinetics of the product.

The advantages of suspension dosage forms include effective dispensing of hydrophobic drugs; avoidance of the use of co-solvents; masking of unpleasant taste of certain ingredients; offering resistance to degradation of drugs due to hydrolysis, oxidation or microbial activity; easy swallowing for young or elderly patients; and efficient intramuscular depot therapy. In addition, when compared to solution dosage forms, relatively higher concentration of drugs can be incorporated into suspension products. To date, numerous theories have been introduced and successfully used to explain the unique behavior of suspension preparations.

An important consideration in any treatment regime is to ensure that the patient receives the correct dose of medicine. For many patients and many drugs there is an acceptable dose window that allows fixed-dose medicines to be used to treat patients with a wide range of body weights without the need to precisely adjust the dose. However, there are other groups of patients where the “fixed-unit-dose” model may not be appropriate, depending on the drug's therapeutic index and pharmacokinetics, e.g. pediatric patients, geriatric patients, patients with severe renal insufficiency and patients with severe hepatic insufficiency. Oral solid unit dose forms, e.g. tablets and capsules, are not convenient under such circumstances since they are fixed strength unit dose forms. In contrast, oral liquid dose forms do have the in-built flexibility that allows the dose to be tailored to the patients' needs.

Where the drug is sufficiently soluble, a solution dosage form, e.g. a simple mixture, may be used. But not all drugs are sufficiently soluble to allow suitable strength solution medicines to be developed and manufactured with an acceptable shelf-life. In such cases, an alternative approach could be to develop a stable aqueous suspension that will allow consistent dosing of the patient. Pharmaceutical suspensions have several advantages and disadvantages when compared to other dosage forms. Since suspensions are liquids, dose adjustment for patients with renal or hepatic impairment, or for pediatric or geriatric patients, may be more straightforward. This is an oversimplification of the development of a dosing strategy for a drug candidate. There are many other details that must be considered for a formulation development project to be successful, but it does provide a simple overview of some of the issues.

The suspension must be physically stable (no appreciable settling) for a sufficient time, chemically stable over the required time (shelf-life), possess a viscosity that allows it to be used for its intended purpose, be easily reconstituted by shaking, and be acceptable in use to the patient, care-giver or other user.

Some materials may possess a combination of properties useful in the formulation and manufacture of stable, elegant pharmaceutical suspensions. Formulation scientists need to consider the totality of properties possessed by a particular excipient. Even though it is being added for one particular characteristic, the other properties will still be present, and will still influence the formulation.

Many of the recently discovered active pharmaceutical ingredients are quite hydrophobic with limited solubility. They may also be quite distasteful. Other drugs may also have quite a high chemical degradation precluding them to be administered as aqueous solutions, and in this case, it may be possible to synthesize an insoluble derivative. In other cases, some drugs are required to be present in the gastrointestinal tract or in the pre-corneal pocket with long residence time. For such drugs, a suspension is an ideal delivery system as it provides better chemical stability and larger surface area and is often more bioavailable than aqueous solutions, tablets, and capsules.

Formulation of an elegant, stable, preserved, safe, and effective suspension is a technically challenging task compared aqueous solutions, tablets, and capsules. Pharmaceutical suspensions are thermodynamically unstable systems. Thus, preparation of such systems is often associated with problems of physical stability, content uniformity, sedimentation, caking, re-suspendibility, and crystal growth. Furthermore, issues related to the masking of bitter taste and undesirable odor of the pharmaceutical ingredient must be taken into consideration.

Some desirable attributes of a suspension are described as follows,

1. It should be safe, effective, stable, and pharmaceutically elegant during the shelf life of the product.

2. The drug should not have a quick sedimentation rate. Furthermore, it should re-suspend easily upon shaking and it must not cake.

3. Physical attributes such as particle size, particle size distribution, viscosity should remain fairly uniform throughout the shelf life of the product.

4. Its viscosity must promote free and uniform flow from the container. The product must have appropriate substantivity that it spreads freely over the affected area.

5. Re-suspension should produce a homogeneous mix of drug particles such that there is a content uniformity with each dose.

A quick means to identify whether or not a drug may be more suitable for solution or suspension is to overlap the pH-stability profile with the pH-solubility profile. This overlap creates a window, which may suggest which dosage form might be most desirable and subsequently the type of excipients needed.

Oral liquid formulation needs a meticulous blend of ingredients to perform various functions like wetting and solubilization, stabilization and to impart suitable color, taste and viscosity. The blend should be compatible, non-reactive and stable. The common excipients generally required for any liquid formulation are vehicles (base), viscosity builders, stabilizers, preservatives, colors and flavors. In addition, solubilizers are required in case of clear liquids, suspending agents are needed for suspensions and emulsifying agents for emulsions.

Cinacalcet is a calcium-sensing receptor agonist. It is commercially available as tablets (Sensipar®) in US since 2004 and is indicated for the treatment of secondary hyperparathyroidism resulting from chronic kidney disease and for the treatment of hypercalcemia in patients with either parathyroid carcinoma or hyperparathyroidism The Sensipar® prescribing information approved by the USFDA suggests that for all indications Sensipar® should always be taken whole and not divided. The present invention, therefore, in one of its principal aspects provides liquid dosage forms of Cinacalcet or pharmaceutically acceptable salt thereof.

The liquid dosage forms according to the present invention include, but not limited to, liquids, liquid dispersions, suspensions, solutions, emulsions, ointments, creams, sprays, spot-on, syrups, elixirs, drops, gels, solution-gels, concentrates and the like. Such liquid dosage forms can be prepared using appropriate one or more pharmaceutically acceptable excipients or additives. Such excipients or additives may be known to those skilled in the art.

Suspensions possess certain advantages over other liquid dosage forms. Some drugs are insoluble in all acceptable media and must, therefore, be administered as a tablet, capsule, or as a suspension. In addition, disagreeable tastes can be masked by a suspension of the drug or a derivative of the drug. Drugs in suspension are chemically more stable than in solution. Therefore, in one of the further aspects, the present invention provides suspension dosage forms of Cinacalcet or pharmaceutically acceptable salt thereof.

Liquid dosage forms are designed as ready to use liquids and as powder for reconstitution into liquid orals like syrups, solutions, suspensions and emulsions. Powder for reconstitution may require skills & expertise and needs to be prepared by a healthcare provider and may not be prepared by the patient or caregiver. The reconstitution process may also be a time consuming process and the patient cannot be benefited by the immediate dose of Cinacalcet as and when required. In such a situation, ready to use, liquid dosage forms of Cinacalcet may be very useful and the patients can be given required doses immediately using ready to use, liquid dosage forms of Cinacalcet. In one of the further aspects, the present invention therefore provides ready to use, liquid dosage forms of Cinacalcet or pharmaceutically acceptable salt thereof.

Liquid dosage forms of an active drug can be prepared using one or more pharmaceutically acceptable excipients or additives suitable for the preparation of liquid dosage forms. In one of the further aspects, the present invention provides liquid dosage forms of Cinacalcet or pharmaceutically acceptable salt thereof and one or more excipients or additives suitable for preparing liquid dosage forms.

The term “pharmaceutically acceptable excipients or additives” as used herein refers to such pharmaceutically acceptable excipients which are known to those skilled in the art for the purposes of preparing liquid dosage forms of the present invention. Such pharmaceutically acceptable excipients, without limitation include, vehicles, solvents/co-solvents, solubilizers, solubility enhancing agents, tonicity agents, permeation/penetration enhancers, mucoadhesives, suspending agents/thickening agents/viscosity modifying agents, bulking agents/auxiliary suspending agents, wetting agents, anti-foaming agents, anti-caking agents, stabilizing agents, anti-oxidants, chelating agents, buffering agents/pH modifying agents/pH adjusting agents, surfactants, preservatives, sweetening agents, flavoring agents and the like or any combination thereof. Such pharmaceutically acceptable excipients can be used in an amount which provides the liquid dosage forms of the present invention desired property for which they are intended or desired to use.

In one of the aspects, the present invention provides liquid dosage forms of Cinacalcet comprising Cinacalcet or pharmaceutically acceptable salt thereof and one or more excipients or additives selected from the group comprising of vehicles, solvents/co-solvents, solubilizers, suspending agents/thickening agents/viscosity modifying agents, anti-foaming agents, anti-caking agents, surfactants, pH adjusting agents and/or pH modifying agents and/or buffering agents or any combination thereof.

Microbiological contamination presents a significant health hazard in oral liquids. Therefore, the use of preservatives become inevitable to prevent the growth of microorganisms during the product's manufacture and shelf life. Therefore, in one of the further aspects, the liquid dosage forms of the present invention may also comprise anti-microbial agents or preserving agents or preservatives.

Increase in the palatability of the drug formulations increases the patient compliance and patient acceptability towards the drug. In one of the further aspects, the present invention therefore provides palatable liquid dosage forms comprising Cinacalcet or pharmaceutically acceptable salts thereof and at least one or both selected from sweeteners/sweetening agents and flavoring agents.

The liquid dosage forms according to the present invention, without limitation include, aqueous dosage forms, alcoholic and/or hydro-alcoholic dosage forms and non-aqueous dosage forms. Aqueous dosage forms according to the present invention may also comprise one or more non-aqueous and/or organic solvents.

In certain aspects, the present invention provides liquid dosage forms of Cinacalcet in the form of suspensions comprising Cinacalcet or pharmaceutically acceptable salt thereof, vehicle(s), solvent(s)/co-solvent(s), solubilizer(s), suspending agent(s)/thickening agent(s)/viscosity modifying agent(s), preservative(s), anti-foaming agent(s), surfactant(s), pH adjusting agent(s)/pH modifier(s) or buffering agent(s) or both, sweetener(s) and flavoring agent(s).

In certain aspects, the present invention provides liquid dosage forms of Cinacalcet in the form of solutions comprising Cinacalcet or pharmaceutically acceptable salt thereof, vehicle(s), solvent(s)/co-solvent(s), solubilizer(s), preservative(s), surfactant(s), pH adjusting agent(s)/pH modifier(s) or buffering agent(s) or both, sweetener(s) and flavoring agent(s).

In one of the further aspects, the liquid dosage forms of the invention may be administered orally or via the oral cavity. The liquid dosage forms of the present invention may also be administered transmucosally, sublingually, via the buccal cavity, via mucosal membranes and/or through the gastrointestinal tract. In one of the further aspects, the liquid dosage forms of the present invention may be administered via pulmonary, intravenous, rectal, opththatmic, colonic, parenteral, intracisternal, intravaginal, intraperitoneal, local, or topical administration.

In some of the aspects, the liquid dosage forms of the present invention are in the form of spray and may be administered by oral route or nasal route. Sprays are known by various names such as aerosol sprays, liquid pump sprays, or activated mists etc.

In some of the aspects, the liquid dosage forms of the present invention are in the form of immediate release dosage forms or modified release dosage forms, such as extended release, controlled release, sustained release, prolonged release and delayed release. In some of the aspects, the liquid dosage forms comprise Cinacalcet or pharmaceutically acceptable salt thereof one or more suitable excipients or additives for the preparation of modified release dosage forms such as rate controlling polymers.

The liquid dosage forms of the present invention may also be prepared by reconstitution of dry powder in suitable diluent or media such as water. The dry powder for reconstitution may be in the form of immediate release forms and comprise Cinacalcet or pharmaceutically acceptable salt thereof and one or more suitable excipients selected form the group comprising of fillers, binders, diluents, disintegrants, pore formers, lubricants, glidants, sweeteners, stabilizing agents, antioxidants, flavoring agents, suspending agents/thickening agents/viscosity modifying agents, surfactants, preservatives and plasticizers. The dry powder for reconstitution may also be in the form of modified release forms and comprise modified release pellets, granules or particles. Such modified release pellets, granules or particles comprise one or more suitable excipients such as rate controlling polymers.

In one of the further aspects, the liquid dosage forms of the invention are suitable for administration to all types of patients' population. In particular, liquid dosage forms of the invention are suitable for pediatric and geriatric patients. The liquid dosage forms of the invention are also useful for the patients who are unable to take solid oral therapy.

In one of the further aspects, the pH of the liquid dosage forms of the present invention is between about 2.0 and about 11.0, preferably between about 3.0 and about 9.0, more preferably between about 4.0 and about 8.0, more preferably between about 5.0 and about 7.0 and more preferably between about 5.5 and about 6.5.

In one of the further aspects, the liquid dosage forms of the present invention are stable for prolonged time when stored under storage conditions. The term“storage conditions” as used herein without limitation include typical storage conditions such as 2° C.-8° C., 40° C.±2° C./75±5% RH, 30° C.±2° C./65±5% RH, 25° C.±2° C./40±5% RH, 25° C.±2° C./60±5% RH, and 40° C.±2° C.NMT 25% RH (NMT=not more than) and accelerated conditions such as 40° C.±2° C./75±5% RH. The term “prolonged time” as used herein indicates that the liquid dosage forms of the present invention are stable for at least 1 month, at least 3 months, at least 6 months or at least 12 months when stored under storage conditions.

As used herein, the terms “stable” or “stability” encompass any characteristic of the liquid dosage forms which may be affected by storage conditions including, without limitation, potency, total impurities, degradation products, specific optical rotation, optical purity, water content, appearance, viscosity, sterility, and colour and clarity. The storage conditions which may affect stability include, for example, duration of storage, temperature, humidity, and/or light exposure.

In some of the aspects of the present invention, stable liquid dosage forms or stability of the liquid dosage forms refer to dosage forms which retain at least about 90%, or about least about 95%, or at least about 96%, or at least about 98%, of the labelled concentration of Cinacalcet or salt thereof contained in the said dosage form after storage under typical and/or accelerated conditions. In further aspects, stable liquid dosage forms or stability of the liquid dosage forms refer to less than about 15% (area percent), or less than about 10% (area percent), or less than about 7% (area percent), or less than about 5% (area percent), or less than about 2% (area percent) of Cinacalcet-related impurities are present after storage under typical and/or accelerated conditions.

In some of the aspects, liquid dosage forms of the present invention contain no more than about 15% (area percent), or no more than about 10% (area percent), or no more than about 7% (area percent), or no more than about 5% (area percent), or no more than about 2% (area percent), or no more than about 1% (area percent), or no more than about 0.5% (area percent), or no more than about 0.2% (area percent), or no more than about 0.1% (area percent) any known or unknown single Cinacalcet-related impurity or other impurity after storage under typical and/or accelerated conditions.

In some of the aspects, liquid dosage forms of the present invention contain no more than about 15% (area percent), or no more than about 10% (area percent), or no more than about 7% (area percent), or no more than about 5% (area percent), or no more than about 2% (area percent), or no more than about 1% (area percent), or no more than about 0.5% (area percent), or no more than about 0.2% (area percent), or no more than about 0.1% (area percent) total Cinacalcet-related impurities or other impurities after storage under typical and/or accelerated conditions.

Methods for determining the stability of the liquid dosage forms of the present invention with respect to a given parameter are well-known to those of skill in the art. For example, individual impurities and total impurities can be assessed by high-performance liquid chromatography (HPLC) or thin layer chromatography (TLC). Unless otherwise indicated to the contrary, a percentage amount of any individual impurities (known/unknown), or total impurities reported herein in the liquid dosage forms are determined by a peak area percent method using HPLC.

The term “comprise/comprises/comprising” as used herein mean that other ingredients, steps, etc. are optionally present. When reference is made herein to a method comprising two or more defined steps, the steps can be carried in any order or simultaneously (except where the context excludes that possibility), and the method can include one or more steps which are carried out before any of the defined steps, between two of the defined steps, or after all of the defined steps (except where the context excludes that possibility).

The term “about,” as used herein, refers to any value which lies within the range defined by a variation of up to 10% of the value.

The use of the terms “a” and “an” and “the” and similar referents in the context of describing the invention (especially in the context of the claims) are to be construed to cover both the singular and the plural, unless otherwise indicated herein or clearly contradicted by context.

All percentages mentioned herein, unless otherwise indicated, are on a w/v basis, i.e. percentage ingredient (active/inactive) present in the total volume of the liquid dosage form.

In accordance with the methods of use and administration of medicinal products, packaging materials, closures and containers vary a great deal and have to meet a wide variety of different requirements. The liquid dosage forms of the present invention may be packaged within any type of pharmaceutically-acceptable package, containers, pumps, bottles with spray pump, bottles with dropper assembly, bottles, collapsible tubes, glass ampoules, stoppered vials, pre-filled syringes, low-density polyethylene (LDPE), high-density polyethylene (HDPE), polyolefin, polypropylene containers and bottles depending upon the quantity of the final dosage form. The bottles and containers without limitation include clear/transparent/opaque or amber colored glass bottles and containers and clear/transparent/opaque or amber colored plastic bottles and containers made from polyethylene, polyamide, polycarbonate, acrylic multipolymers, polypropylene, polyethylene terephthalate, polyvinyl chloride, polystyrene and the like. Depending upon the type of the containers or bottles, closures may have different shapes and sizes. The closure of the packaging material may be made from polyethylene, polyamide, polycarbonate, acrylic multipolymers, polypropylene, polyethylene terephthalate, polyvinyl chloride, polystyrene and the like.

Liquid dosage forms of the present invention may be packaged in a sterile single use bottle/container that contains a unit dose for administration to a patient. Suitable bottles/containers may contain volumes between 1-10 ml, 10-20 ml, 20-40 ml, and 40-100 ml, and even more. The container may typically comprise Cinacalcet or pharmaceutically acceptable salt thereof in an amount of between 10-40 mg, between 40-80 mg, between 80-130 mg, and even more. Thus, it may also be noted that the container may be a multi-use container (i.e., retains at least one more unit dose after a first unit dose is dispensed).

Following embodiments of the invention describe suitable excipients which may be used to prepare liquid dosage forms of the present invention. It is in no way the intention of the present inventor(s)/applicant(s) to limit the scope of the liquid dosage forms of the present invention by the description of following embodiments. Described embodiments are for illustrative purpose only and a skilled person may use other excipients from the same or different classes as well which may provide liquid dosage forms of the present invention same or improved physico-chemical properties, palatability, stability and the like and retain or increase patients' acceptability towards the therapy. Such other excipients, classes of excipients and compositions resulted therefrom are also part of the present invention and covered within the scope of the present invention.

Vehicles may be used in the liquid compositions of the present invention. Vehicles are the liquid bases that carry drugs and other excipients in dissolved or dispersed state. Vehicles may be aqueous or non-aqueous or mixture thereof. Non-aqueous solvents/co-solvents may also be added in the liquid compositions of the present invention to increase the solubility of poorly soluble substances and enhance the chemical stability of a drug. Suitable solvents/co-solvents, solubilizers or vehicles, that may be employed, in the liquid compositions of the invention include, but are not limited to, dichloromethane, acetonitrile, ethyl acetate, acetone, propylene carbonate, water, glycerine, coconut fatty acid diethanolamide, medium and/or long chain fatty acids or glycerides, monoglycerides, diglycerides, triglycerides, structured triglycerides, soyabean oil, peanut oil, corn oil, corn oil monoglycerides, corn oil diglycerides, corn oil triglycerides, polyethylene glycol, capryocaproylmacroglycerides, caproyl 90, propylene glycol, polyoxyethylenesorbitan fatty acid esters, polyoxyethylene castor oil derivatives, castor oil, cottonseed oil, olive oil, safflower oil, peppermint oil, coconut oil, palm seed oil, beeswax, oleic acid, methanol, ethanol, isopropyl alcohol, butanol, acetone, methyl isobutyl ketone, methyl ethyl ketone and the like or any combinations thereof.

Wetting agents as used herein are routinely used in pharmaceutical formulations, especially in liquid dosage forms to create a homogeneous dispersion of solid particles in a liquid vehicle. This process can be challenging due to a layer of adsorbed air on the particle's surface. Hence, even particles with a high density may float on the surface of the liquid until the air phase is displaced completely. The use of a wetting agent allows removal of adsorbed air and easy penetration of the liquid vehicle into pores of the particle in a short period of time. For an aqueous vehicle, alcohol, glycerin, and PG are frequently used to facilitate the removal of adsorbed air from the surface of particles. Whereas for a non-aqueous liquid vehicle, mineral oil is commonly used as a wetting agent. Non-limiting examples of wetting agents are Benzalkonium chloride, Benzethonium chloride, Cetylpyridinium chloride, Docusate sodium, Nonoxynol 9, Octoxynol, Poloxamer, Poloxamer 124, Poloxamer 188, 237, 338, 407, Polyoxyl 35 castor oil, Polyoxyl 40 hydrogenated castor oil, Polyoxyl 10 oleyl ether, Polyoxyl 20 cetylstearyl ether, Polyoxyl 40 stearate, Polysorbate 20, Polysorbate 40, Polysorbate 60, Polysorbate 80, Sodium lauryl sulfate, Sorbitan monolaurate, Sorbitan monooleate, Sorbitan monopalmitate, Sorbitan monostearate, Tyloxapol and the like or any combinations thereof.

Solubility enhancing agents may include, but are not limited to, DL-methionine, caffeine, nicotinamide, vanillin, benzyl alcohol, ethanol and diethylene glycol monoethyl ether and the like or combinations thereof.

Stabilizing agents may include, but are not limited to, sodium metabisulphite, sodium bisulphite, ethylene diamine tetraacetic acid (EDTA) or salts thereof, ascorbic acid and the like or combinations thereof.

Penetration/permeation enhancers may include, but are not limited to, nicotinamide, caffeine, peppermint oil, sodium glycocholate, phospholipids, alkyl saccharides, aprotinin, benzalkonium chloride, ceramides, cetylpyridinium chloride, chitosan, chitosan-4-thiobutylamidine, cyclodextrins, dextran sulfate, dodecyl azacycloheptyl-2-ketone, ether lipids (plasmologens), glycerol, glycosylated sphingosines, lauric acid, 23-lauryl ether, lysophosphatidyl choline, menthol, methoxysalicylate, phosphatidy choline, 1-pamitoyl-2-ghtaroyl-sn-glycero-3-phosphocholine, polycarbophil cysteine, poly-L-arginine, polyoxyethylene, polyoxyethylene-9-lauryl ether, polysorbate 80, propylene glycol, EDTA, sodium deoxycholate, sodium glycocholate, sodium glycodeoxycholate, sodium lauryl sulfate, sodium salicylate, sodium taurocholate, sodium taurodeoxycholate, sodium taurodihydrofusidate, sphingolipids, sterols and the like or combinations thereof.

Mucoadhesives may also be added in the compositions of the present invention. Examples of suitable mucoadhesives include, but are not limited to, hydroxypropyl cellulose, gelatin, crosslinked polyacrylic acid, polymethacrylic acid, polyhydroxyethyl methacrylic acid, hydroxypropyl methyl cellulose, polyethylene glycol, sodium carboxymethyl cellulose, hyaluronic acid, chitosan, polycarbophil, pectin, xanthan gum, alginate, copolymers of dextran, polyacrylamide, acacia, copolymer of caprolactone and ethylene oxide, carbopol 934, tragacanth, eudragit and the like or combinations thereof.

Stabilizing agents may include, but are not limited to, sodium metabisulphite, sodium bisulphite, ethylene diamine tetraacetic acid (EDTA) or salts thereof, ascorbic acid and the like or combinations thereof.

The pH of an oral liquid formulation is a key point in many regards. Control of the formulation pH, could prevent large changes during storage. Therefore, most formulations utilize a buffer to control potential changes in the solution pH. The amount of buffer capacity needed is generally between 0.01 and 0.1 M, and a concentration between 0.05 and 0.5 M is usually sufficient. The selection of a suitable buffer should be based on (i) Whether the acid-base forms are listed for use in oral liquids, (ii) The stability of the drug and excipients in the buffer, and (iii) The compatibility between the buffer and container. A combination of buffers can also be used to gain a wider range of pH compared to the individual buffer alone. However, not all buffers are suitable for use in oral liquids. For example, a boric acid buffer may be used for optical and IV delivery but not in oral liquids because of its toxicity. The stabilizing effect of buffers that have multiple charged species in solution could also determine the potential reaction between excipients and API. For example, buffers that use carbonates, citrate, tartrate, and various phosphate salts may precipitate with calcium ions by forming sparingly soluble salts. However, this precipitation is dependent upon the solution pH. The activity of phosphate ions may be lowered due to interactions with other solution components.

There are a number of factors that may also affect the solution pH such as temperature, ionic strength, dilution, and the amount and type of co-solvents present. For example, the pH of acetate buffers is known to increase with temperature, whereas the pH of boric acid buffers decreases with temperature. Finally, the drug in solution may itself act as a buffer. If the drug is a weak electrolyte, such as salicylic acid or ephedrine, the addition of base or acid, respectively, will create a system in which the drug can act as a buffer.

One of the most crucial factors involved in formulating a pharmaceutical suspension is the selection of an appropriate suspending agent. Suspending agents impart viscosity, and thus retard particle sedimentation. Other factors considered in the selection of the appropriate agent include desired rheological property, suspending ability in the system, chemical compatibility with other excipients, pH stability, length of time to hydrate, batch-to-batch reproducibility, and cost. Non-limiting examples of pH adjusting agents/modifiers and buffers are Acetic acid, Adipic acid, Ammonium carbonate, Ammonium hydroxide, Ammonium phosphate, Boric acid, Citric acid, Diethanolamine, Fumaric acid, Hydrochloric acid, Malic acid, Nitric acid, Propionic acid, Potassium acetate, Potassium bicarbonate, Potassium chloride, Potassium citrate, Potassium metaphosphate, Potassium phosphate, Sodium acetate, Sodium bicarbonate, Sodium borate, Sodium carbonate. Sodium chloride, Sodium citrate, Sodium glycolate, Sodium hydroxide, Sodium lactate, Sodium phosphate, Sodium proprionate, Succinic acid, Sulfuric acid, Tartaric acid, Triethylamine, Triethanoamine, Tromethamine, Trolamine and the like or any combinations thereof.

Suspending agents can be classified into cellulose derivatives, clays, natural gums, and synthetic gums. In many cases, these excipients are used in combination. There are many water soluble hydrocolloids that can act as suspending agents in the formulation of pharmaceutical suspensions. They can be of natural, semi-synthetic or synthetic origin. Non-limiting examples of suspending agents are Acacia, Agar, Alginic acid, Carbomer, Carmellose sodium, Dextrin, Gelatin, Veegum or Gel white, Gellan gum, Sodium alginate, Methylcellulose, Hydroxyethyl cellulose, Hydroxypropyl cellulose, Hydroxypropylmethyl cellulose, Hydroxypropyl starch, Hypromellose, Malhodextrin, Methylcellulose, Modified starch, Pectin, Poloxamer, Polycarbophil, Polyethylene glycol, Polyvinyl acetate, Poly (vinyl alcohol), Potassium alginate, Polyvinyl pyrrolidone, Pregelatinized starch. Propylene glycol alginate, Sodium alginate, Carboxymethyl cellulose or an alkali metal salt thereof, Microcrystalline cellulose, gum Arabic, Karaya gum, Sterculia gum, Tragacanth, Xanthangum, Bentonite, Carageenan, Guar gum, Colloidal silicon dioxide and the like or any combinations thereof.

Microbiological contamination presents a significant health hazard in oral liquids. Therefore, the use of preservatives become inevitable to prevent the growth of microorganisms during the product's manufacture and shelf life, although it may be most desirable to develop a “preservative-free” formulation to address the increasing concerns about the biological activity of these compounds. Most formulations require some kind of preservative to ensure no microbial growth.

The majority of preservatives are bacteriostatic rather than bacteriocidal, and consists of both acid and nonacid types. Among the acidic types are phenol, chloro-cresol, 9-phenyl phenol, alkyl esters of para-hydroxybenzoic acid, benzoic acid, boric acid, and sorbic acid, and their respective salts. Therefore, the pH of solution, and the pKa of the preservative need to be carefully evaluated prior to selecting a preservative for a formulation. Neutral preservatives include chlorobutanol, benzyl alcohol, and beta-phenylethyl alcohol. Under alkaline conditions, it is generally regarded that microbial growth is insignificant and at these pH values, the need for a preservative is not generally recommended.

Many preservatives listed in the FDA inactive ingredient guide for liquid dosage forms. Unfortunately, many of them are not recommended for use in oral liquids and hence the choice of an acceptable preservative for an oral liquid formulation is limited. In addition, the solubility of many preservatives in aqueous system may not be high enough for effective antimicrobial activity. Additionally, it is essential to understand that bacteriostatic agents like para hydroxyl benzoic acids can partition between organic and aqueous phases in a heterogenous liquid formulations in such a way that their activity is significantly reduced. Non-limiting examples of preservatives are Alcohol, Ethanol, Chlorobutanol, Phenoxyethanol, Potassium benzoate, Benzyl alcohol, Benzoic acid, Potassium sorbate, Sorbic acid, Benzalkonium chloride, Benzethonium chloride, Cetrimonium bromide, Cetylpyridinium chloride, Bronopol, Chlorbutol, Chlorocresol, Cresol, Butylparaben, Methylparaben, Propylparaben, Ethylparaben. Phenol, Thymol, Phenylethanol, Sodium benzoate, Antimicrobial solvents like Propylene glycol, Glycerin, Chloroform and the like or any combinations thereof. In addition, some formulation ingredients like nonionic surfactants, quaternary ammonium compounds, gelatin, ferric salts, calcium salts and salts of heavy metals, including silver, lead, and mercury prevent microbial growth.

Antioxidants can be compounds that can reduce a drug that has been oxidized, or compounds that are more readily oxidized than the agents they are to protect (oxygen scavengers). Many of the lipid-soluble antioxidants act as scavengers. Antioxidants can also act as chain terminators, reacting with free radicals in solution to stop the free-radical propagation cycle. Mixtures of chelating agents and antioxidants are often used because there appears to be a synergistic effect. This occurs because many of the agents act at differing steps in the oxidative process.

Some substances prone to oxidation include unsaturated oils/fats, compounds with aldehyde or phenolic groups, colors, flavors, sweeteners, plastics and rubbers, the latter being used in containers for products. Oxidation may manifest as products with an unpleasant odour, taste, appearance, precipitation, discoloration or even a slight loss of activity. The term rancidity refers to many typical off-flavors that result from autoxidation of unsaturated fatty acids that are present in oils and fats, and it affects many oils and fats. The distinct rancid odour may result from short-chain, volatile monomers resulting from the cleavage of the longer chain, less volatile oils and fats. Non-limiting examples of anti-oxidants are α-Tocopherol acetate, Ascorbic acid, Erythorbic acid, Butylated hydroxytoluene (BHT), d-α-Tocopherol natural, Monothioglycerol, Sodium bisulfite, Sodium sulfite, Sodium metabisulfite, Potassium metabisulfite, Acetone sodium bisulfite, Ascorbyl palmitate, Cysteine, d-α-tocopherol synthetic, Nordihydroguaiaretic acid, Sodium formaldehyde sulfoxylate, Sodium thiosulfate, Acetylcysteine, Ascorbyl palmitate, Butylated hydroxyanisole (BHA), Cysteine hydrochloride, Dithiothreitol, Propyl gallate, Thiourea and the like or any combinations thereof.

In some instances, there are insufficient drug particles in a unit dose of suspension to make a pharmaceutically elegant suspension. This is particularly true for the more highly active drugs, where the unit dose is small. Under such circumstances, the formulator will need to add more particles to improve the appearance of the final product, and also to help stabilize the suspension. To serve this purpose, bulking agents, also known as auxiliary suspending agents are used. Non-limiting examples of bulking agents are Calcium carbonate, Calcium hydroxide, Cellulose, Crospovidone, Dibasic calcium phosphate, Magnesium carbonate, Magnesium hydroxide, Microcrystalline cellulose, Silica (silicon dioxide), Titanium dioxide and the like or any combinations thereof.

Many different materials are capable of adsorbing onto the suspended particles, e.g. natural gums, cellulosics and non-ionic surfactants. However, not all of them are able to act as protective colloids and provide steric hindrance to caking at a sufficiently low concentration. High levels of surfactants, for example, can increase gastro-intestinal motility. Higher molecular weight gums and cellulosics may also cause an unacceptable increase in the viscosity of the system. There are, however, certain polymers, or grades of polymers, that are capable of acting as protective colloids at concentrations that do not markedly increase the viscosity of the system, or increase gut motility, etc. Such materials include poloxamers, lower molecular weight grades of povidone, and low molecular weight grades of some other hydrophilic colloids.

Surfactant is a general name for materials that possess surface activity; in solution they tend to orient at the surface of the liquid. There are several general classes of surfactants: anionic, cationic, amphoteric and non-ionic. Surfactants are amphiphilic molecules, i.e. part of the molecule is hydrophilic, and part is lipophilic. This combination of the two opposite affinities in the same molecule causes them to orient to the interface and thereby reduce the interfacial tension between the continuous and disperse phases, such as in emulsions and suspensions. Ionic surfactants work primarily through electrostatic forces, whereas non-ionic surfactants work primarily through steric forces. Non-limiting examples of surfactants are Sodium lauryl sulfate, Docusate sodium, Cocamidopropyl amino betaine, Polyoxyethylene sorbitan fatty acid esters (Polysorbate, Tween®), Polyoxyethylene 15 hydroxystearate (Macrogol 15 hydroxystearate, Solutol HS 5W), Polyoxyethylene castor oil derivatives (Cremophor® EL, ELP, RH 40), Polyoxyethylene stearates (Myrj®), Sorbitan fatty acid esters (Span®), Polyoxyethylene alkyl ethers (Brij-®), Polyoxyethylene nonylphenol ether (Nonoxynol®) and the like or any combinations thereof.

Anti-foaming agents may be used in the preparation of the liquid pharmaceutical compositions of the present invention to lower the surface tension and cohesive binding of liquid phase. Non-limiting examples of anti-foaming agents are simethicone, organic phosphates, alcohols, paraffin oils, stearates, glycols and the like or any combinations thereof.

Chelating agents, also known as sequestrants, are molecules that have the ability to form stable complexes with metal ions, particularly di-valent and tri-valent metal ions including trace metals and heavy metals. These metal ions are often implicated in API degradation by acting as catalysts, e.g. Mg²⁺ will catalyze both ester hydrolysis and the Maillard interaction between primary or secondary amines and reducing sugars. Oxidative degradation is also often catalyzed by heavy metals. In addition, certain trace metals are required for microbial growth, and chelation (sequestration) to form complexes can help prevent microbial growth and spoilage, and thus allow lower levels of microbiocidal agents to be used. Non-limiting examples of chelating agents are Calcium disodium edetate, Disodium edetate, Edetic acid (also known as ethylenediaminetetracetic acid/EDTA), Citric acid and the like or any combinations thereof.

Palatability of oral medicines is an important factor in compliance. There are several components to palatability including flavor, mouth-feel and sweetness. Most patients prefer medicines that are not too bitter but may be slightly “tart” (acidic). Most APIs are bitter. However, for bitterness to develop, the drug must be sufficiently soluble to interact with taste receptors on the tongue. For insoluble APIs in the form of suspensions, components of the suspension are also bitter, e.g. preservatives, or very salty, e.g. buffer systems. However, a slight saltiness and a slight bitterness are desirable for palatability.

Traditionally, oral medicines were sweetened using Syrup (concentrated sucrose solution) or honey (contains fructose). However, these materials are inadequate for the formulation of many products because they simply are not able to adequately mask the very bitter taste of many pharmaceutical materials, including APIs and excipients. Several alternative sweetening agents have been developed over the years to better mask unpleasant tastes in both processed foods and pharmaceuticals.

Several of the materials classified as sweetening agents are sugar alcohols (also known as polyhydric alcohols, polyols and hydrogenated sugars). Several of the commonly used sweetening agents are ionic and have the potential to interact with other components of the suspension. Some sweetening agents are more stable than others in aqueous solution. These will be important factors in the final selection of the sweetening agent. Non-limiting examples of sweetening agents are Glucose, Sucralose, Trehalose, Fructose, Xylose, Dextrose, Galactose, Tagatose, Maltose, Sucrose, Glycerol, Dulcitol, Mannitol, Lactitol, Sorbitol, Xylitol, Saccharine or the corresponding sodium, potassium or calcium salt, Cyclamate or the corresponding sodium or calcium salt, Aspartame, or Acesulfame or the potassium salt thereof, Dulcin or Ammonium glycyrrhizinate, Alitame, Inulin, Isomalt, Neohesperidin dihydrochalcone, Thaumatin and the like or any combinations thereof.

Flavors are used to improve the palatability of oral medicines. One problem that can arise with oral suspensions is that the suspension may produce a “cloying” sensation in the mouth. While this is not the same as a bitter taste, it can nevertheless cause problems for the patient and affect compliance. This can be a particular problem with high levels of inorganic components. Flavors can help reduce this “cloying” taste and thereby improve palatability, and ultimately patient compliance.

There are many different flavors, and most flavors are complex mixtures of many components. Today most flavors are developed by specialist flavor houses, and typically the flavor is formulated for each individual application. Since flavor will be part of the suspension continuous phase, it has the maximum potential for interaction, and some flavor components may cause stability issues (physical or chemical) for the suspension. Flavor development and compounding is a specialist discipline. When deciding on which particular flavor is appropriate, the flavor specialist would benefit from knowledge of the other likely components in the suspension, just as the formulation scientist would benefit from knowledge of the components of the flavor. Flavors can adsorb onto finely divided solids, thus reducing their effectiveness. They can also be absorbed by packaging. Flavor preferences vary with age, but the citrus flavors appear generally acceptable to most age groups. Non-limiting examples of flavoring agents are synthetic flavor oils and flavoring aromatics and/or natural oils, extracts from plants leaves, flowers, fruits, and so forth and the like or any combinations thereof. These may include cinnamon oil, oil of wintergreen, peppermint oils, clove oil, bay oil, anise oil, eucalyptus, thyme oil, cedar leaf oil, oil of nutmeg, oil of sage, oil of bitter almonds, and cassia oil and the like or any combinations thereof. Also useful as flavors are vanilla, citrus oil, including lemon, orange, grape, lime and grapefruit, and fruit essences, including apple, banana, pear, peach, strawberry, raspberry, cherry, plum, pineapple, apricot, and so forth and the like or any combinations thereof. Solid forms, such as spray dried forms of flavoring agents, may also be useful in the liquid dosage forms disclosed herein.

Coloring agents may also be used in the preparation of the liquid compositions of the present invention. Pharmaceutical colors come in two types; soluble dyes and insoluble pigments. For pharmaceutical suspensions intended for oral use, soluble dyes are often used; however, pigments may also be used and would be part of the disperse phase. Soluble dyes have the potential to interact with other components of the formulation.

In some of the aspects, the liquid dosage forms of the present invention are non-caking liquid dosage forms. The term “non-caking” as used herein means that the liquid dosage form has a smooth consistency and doesn't contain any caking or clumping particles, by visual inspection. Also, the liquid dosage form in accordance with the present invention does not cake or clump during manufacture, i.e., when mixed with excipients. Nor does it cake or clump upon storage, even under relatively humid conditions, e.g., a relative humidity of about 75% or greater and when stored for relatively long periods such as about 6 months or longer and even at elevated temperatures of about 40° C. or greater, or at any combination of such humidity, time and temperature parameters. Thus, the liquid dosage forms in accordance with the present invention will remain non-caking during typical storage and use conditions.

“Cinacalcet” as used herein, unless the context requires otherwise, includes Cinacalcet, its pharmaceutically acceptable salts and chemical derivatives thereof such as polymorphs, solvates, hydrates, anhydrous forms, amorphous forms, prodrugs, chelates, and complexes. “Cinacalcet” as used herein also includes racemic or substantially pure forms.

In one of the further aspects, Cinacalcet or pharmaceutically acceptable salt thereof used for the preparation of the liquid dosage forms of the present invention comprise particles of Cinacalcet or pharmaceutically acceptable salt thereof, wherein the d₉₀ of the particles of Cinacalcet or pharmaceutically acceptable salt thereof is less than about 1000 μm, or less than about 950 m, or less than about 900 μm, or less than about 850 μm, or less than about 800 μm, or less than about 750 μm, or less than about 700 μm, or less than about 650 μm, or less than about 600 μm, or less than about 550 μm, or less than about 500 μm, or less than about 450 μm, or less than about 400 μm, or less than about 350 μm, or less than about 300 μm, or less than about 250 μm, or less than about 200 μm, or less than about 150 μm, or less than about 100 μm, or less than about 90 μm, or less than about 80 μm, or less than about 70 μm, or less than about 60 μm, or less than about 50 μm, or less than about 40 μm, or less than about 30 μm, or less than about 20 μm, or less than about 10 μm, or less than about 5 μm, or less than about 2 μm, or less than about 1 μm, or less than about 0.5 μm.

In one of the further aspects, the liquid dosage forms of the present invention comprise particles of Cinacalcet or pharmaceutically acceptable salt thereof, wherein the d₉₀ of the particles of Cinacalcet or pharmaceutically acceptable salt thereof is less than about 1000 μm, or less than about 950 μm, or less than about 900 μm, or less than about 850 μm, or less than about 800 μm, or less than about 750 μm, or less than about 700 μm, or less than about 650 μm, or less than about 600 μm, or less than about 550 μm, or less than about 500 μm, or less than about 450 μm, or less than about 400 μm, or less than about 350 μm, or less than about 300 μm, or less than about 250 μm, or less than about 200 μm, or less than about 150 μm, or less than about 100 μm, or less than about 90 μm, or less than about 80 μm, or less than about 70 μm, or less than about 60 μm, or less than about 50 μm, or less than about 40 μm, or less than about 30 μm, or less than about 20 μm, or less than about 10 μm, or less than about 5 μm, or less than about 2 μm, or less than about 1 μm, or less than about 0.3 μm.

In one of the aspects, general formula of the liquid dosage forms according to the present invention may be provided as follows.

TABLE 1
General formula of liquid dosage forms of the present invention

| | | Quantity (% w/v) | |
|---|---|---|---|
| | | Solution | Suspension |
| Sr No | Ingredient | dosage form | dosage form |
| 1 | Cinacalcet or pharmaceutically | 0.01-25 | 0.01-25 |
| | acceptable salt thereof (active | | |
| | ingredient) | | |
| 2 | Suspending agent(s)/thickening | — | 0.01-10 |
| | agent(s)/viscosity modifying agent(s) | | |
| 3 | Preservative(s) | 0.01-10 | 0.01-10 |
| 4 | Wetting agent(s) | — | 0-90 |
| 5 | pH adjusting agent(s)/pH modifying | Q.S. to | Q.S. to |
| | agents | adjust the pH | adjust the pH |
| 6 | Buffering agent(s) | Q.S. to | Q.S. to |
| | | adjust the pH | adjust the pH |
| 7 | Solvent(s)/co-solvent(s) | Q.S. | Q.S. |
| 8 | Solubilizer(s) | Q.S. | Q.S. |
| 9 | Anti-foaming agent(s) | — | 0.01-10 |
| 10 | Anti-caking agent(s) | — | 0-10 |
| 11 | Antioxidant(s) | — | 0-10 |
| 12 | Surfactant(s) | 0-10 | 0.01-10 |
| 13 | Sweetening agent(s) | 0.01-5 | 0.01-5 |
| 14 | Flavoring agent(s) | 0.01-5 | 0.01-5 |
| 15 | Coloring agent(s) | 0-2 | 0-2 |
| 16 | Vehicle(s) | Q.S. | Q.S. |

Q.S. = Quantity Sufficient

Those who are skilled in the art will appreciate that different types of liquid dosage forms as described herein can be prepared by using suitable excipients or additives known in the art. Thus, the name of excipients or additives and proportionate range thereof provided in the Table-1 is provided herein for the illustration purpose only and should not be construed as the exact or the only scope of the present invention. The liquid dosage forms of the present invention may be prepared using suitable excipients or additives in any suitable amount.

In one of the further aspects, the present invention provides process for the preparation of the liquid dosage forms of Cinacalcet or pharmaceutically acceptable salt thereof.

The general process for preparing liquid dosage forms of the present invention can be described as follows.
(a) One or more preservatives are added in the sufficient quantity of the vehicle;
(b) One or more sweetener, optionally one or more antifoaming agents, optionally one or more surfactants, one or more solvents/co-solvents or solubilizers are sequentially added;
(c) Cinacalcet or pharmaceutically acceptable salt thereof is added;
(d) Optionally one or more suspending agents/thickening agents/viscosity modifying agents, one or more pH adjusting agents and/or pH modifying agents and/or buffering agents (to adjust the pH) and one or more flavoring agents and/or one or more solvents/co-solvents are added sequentially; and
(e) Required quantity of vehicle is added to make up the volume to the final quantity.

Those who are skilled in the art can understand that some variations in the process described herein can be adopted. A skilled person may omit use of some pharmaceutical excipients as described herein above. A skilled person may also alternatively use some or all pharmaceutical excipients as described herein from the same excipient classes. Such variations are well within the scope of the present invention. A skilled person can also change and/or omit steps of their sequences of the herein described process for the purposes of suitability and convenience where one or more pharmaceutically acceptable excipients may or may not be used without affecting and diminishing the quality and characteristics of the resulting product. Such variations/changes/omissions/additions are well within the scope of the present invention.

The liquid dosage forms of the present invention may also be prepared using processes generally known to those skilled in the art. The processes for the preparation of liquid dosage forms of the present invention may vary depending upon the final dosage form, e.g. solution, suspension, etc. The processes for the preparation of the liquid dosage forms of the present invention may comprise multiple steps. Such steps may include sequential addition of suitable excipients/additives. Such steps may also include physical processes for example mixing, stirring, agitation etc.

In one of the aspects, the liquid dosage forms of the present invention are suitable for administration to a subject to treat or prevent a disease or condition. Preferably, the subject is a mammal. More preferably, the mammal is a human. Preferably, the disease or condition is a disease or condition that is treatable by the administration of Cinacalcet or pharmaceutically acceptable salt thereof.

In one of the aspects, the present invention is directed to the method for the treatment of a disease or disorder or medical condition that can be treated by altering a subject's calcium receptor activity. In one of the further aspects, the present invention is directed to the method for the treatment of a disease or disorder or medical condition chosen from hyperparathyroidism, such as primary hyperparathyroidism and secondary hyperparathyroidism, hyperphosphonia, hypercalcemia, and elevated calcium-phosphorus product comprising administering to a patient, such as human, an effective dosage amount of a liquid dosage form comprising Cinacalcet or pharmaceutically acceptable salt thereof and one or more pharmaceutically acceptable excipient or additives as disclosed herein.

In one of the further aspects, the present invention is directed to the method for the treatment of a disease or disorder or medical condition chosen from osteoporosis, arterial stiffness, anemia, familial hypophosphatemic rickets, hemodialysis, parathyroid tumors, vascular diseases, recurrent prostate cancer, adenocarcinoma of prostate, parathyroid adenoma, parathyroid hyperplasia, renal osteodystrophy, osteomalacia, memory functions, familial primary hyperparathyroidism, parathyroid neoplasms, coronary artery calcification and cardiovascular diseases comprising administering to a patient, such as human, an effective dosage amount of a liquid dosage form comprising Cinacalcet or pharmaceutically acceptable salt thereof and one or more pharmaceutically acceptable excipient or additives as disclosed herein.

“Effective dosage amount” as used herein with respect to, for example Cinacalcet liquid dosage forms shall mean that dosage that provides the specific pharmacological response for which Cinacalcet administered in a significant number of subjects in need of such treatment. It is emphasized that “effective dosage amount” administered to a particular subject in a particular instance will not always be effective in treating the diseases described herein, even though such dosage is deemed a “effective dosage amount” by those skilled in the art.

In one of the further aspects, the present invention is directed to use liquid dosage forms of Cinacalcet or pharmaceutically acceptable salt thereof prepared according to the present invention for the treatment of a disease or disorder or medical condition that can be treated by altering a subject's calcium receptor activity. In one of the further aspects, the present invention is directed to use the liquid dosage forms of Cinacalcet or pharmaceutically acceptable salt thereof prepared according to the present invention for the treatment of a disease or disorder or medical condition chosen from hyperparathyroidism, such as primary hyperparathyroidism and secondary hyperparathyroidism, hyperphosphonia, hypercalcemia, and elevated calcium-phosphorus product.

In one of the further aspects, the present invention is directed to use liquid dosage forms of Cinacalcet or pharmaceutically acceptable salt thereof prepared according to the present invention for the treatment of a disease or disorder or medical condition chosen from osteoporosis, arterial stiffness, anemia, familial hypophosphatemic rickets, hemodialysis, parathyroid tumors, vascular diseases, recurrent prostate cancer, adenocarcinoma of prostate, parathyroid adenoma, parathyroid hyperplasia, renal osteodystrophy, osteomalacia, memory functions, familial primary hyperparathyroidism, parathyroid neoplasms, coronary artery calcification and cardiovascular diseases.

The liquid dosage forms of the present invention are proposed to have unexpectedly dramatic dissolution profiles. Rapid dissolution of an administered active agent is preferable, as faster dissolution generally leads to greater bioavailability and faster onset of action. To improve the dissolution profile and biovailability of Cinacalcet it would be useful to increase Cinacalcet's dissolution so that it could attain a level close to 100% dissolution of the drug substance.

The liquid dosage forms of the present invention comprising Cinacalcet or a pharmaceutically acceptable salt thereof, exhibit improved or comparable pharmacokinetic profiles as compared to known Cinacalcet compositions, e.g. Sensipar®, For example, the Cmax and/or AUC of the liquid dosage forms of Cinacalcet of the present invention can be greater than or substantially equal to the Cmax and/or AUC for known Cinacalcet compositions administered at the same dosage. In addition, the Tmax of the liquid dosage forms of Cinacalcet of the present invention can be lower than or substantially equal to that obtained for a known Cinacalcet compositions, administered at the same dosage. In addition, combinations of an improved or comparable Cmax, AUC and Tmax profile can be exhibited by the liquid dosage forms of Cinacalcet of the invention, as compared to known Cinacalcet compositions. In further aspects, the liquid dosage forms of Cinacalcet of the present invention may result in minimal different absorption levels when administered under fed as compared to fasting conditions.

In one of the aspects, a liquid dosage form comprising Cinacalcet or pharmaceutically acceptable salt thereof exhibits in comparative pharmacokinetic testing with an Cinacalcet marketed or known formulation, administered at the same dose, a Tmax not greater than about 90%, not greater than about 80%, not greater than about 70′, not greater than about 60%, not greater than about 50%, not greater than about 30%, not greater than about 25%, not greater than about 20%, not greater than about 15%, not greater than about 10%, or not greater than about 5% of the Tmax exhibited by the marketed or known Cinacalcet formulation.

In one of the further aspects, the liquid dosage form comprising Cinacalcet or pharmaceutically acceptable salt thereof exhibits in comparative pharmacokinetic testing with an Cinacalcet marketed or known formulation, administered at the same dose, a Cmax which is at least about 50%, at least about 100%, at least about 200%, at least about 300%, at least about 400%, at least about 500%, at least about 600%, at least about 700%, at least about 800%, at least about 900%, at least about 1000%, at least about 1100%, at least about 1200%, at least about 1300%, at least about 1400%, at least about 1500%, at least about 1600%, at least about 1700%, at least about 1800%, or at least about 1900% greater than the Cmax exhibited by the marketed or known Cinacalcet formulation.

In one of the further aspects, the liquid dosage form comprising Cinacalcet or pharmaceutically acceptable salt thereof exhibits in comparative pharmacokinetic testing with an Cinacalcet marketed or known formulation, administered at the same dose, an AUC which is at least about 25%, at least about 50%, at least about 75%, at least about 100%, at least about 125%, at least about 150%, at least about 175%, at least about 200%, at least about 225%, at least about 250% a, at least about 275%, at least about 300%, at least about 350%, at least about 400%, at least about 450%, at least about 500%, at least about 550%, at least about 600%, at least about 750%, at least about 700%, at least about 750%, at least about 800%, at least about 850%, at least about 900%, at least about 950%, at least about 1000%, at least about 1050%, at least about 1100%, at least about 1150%, or at least about 1200% greater than the AUC exhibited by the marketed or known Cinacalcet formulation.

In one of the further aspects, the Tmax of Cinacalcet or salt thereof, when assayed in the plasma of the mammalian subject, is less than about 6 to about 8 hours. In other aspects of the invention, the Tmax of Cinacalcet or salt thereof is less than about 6 hours, less than about 5 hours, less than about 4 hours, less than about 3 hours, less than about 2 hours, less than about 1 hour, or less than about 30 minutes after administration.

In some aspects, the liquid dosage forms of Cinacalcet of the present invention exhibit improved or comparable bioavailability as compared to known Cinacalcet compositions, e.g. Sensipar®.

The present invention is further exemplified by the following non-limiting examples.

### BEST MODE OF CARRYING OUT THE INVENTION

### Examples

The liquid dosage forms of the present invention are explained in more detail with reference to the following examples. These examples are provided by way of illustration only and should not be construed as to limit the scope or spirit of the claims in any manner.

### Example-1: Preparation of Suspension Dosage Form of Cinacalcet

| Sr No | Name of Ingredient | mg/mL |
|---|---|---|
| 1 | Cinacalcet hydrochloride | 10-40 |
| 2 | Hydroxypropyl methyl | 0.1-2.5 |
| | cellulose (HPMC) | |
| 3 | Sucralose | 0.5-5 |
| 4 | Glycerin | Q.S. |
| 5 | Methyl paraben | 0.1-2 |
| 6 | Propyl paraben | 0.1-2 |
| 7 | Simethicone | 0.1-5 |
| 8 | Fraise flavor | 0.05-2 |
| 9 | Polysorbate 80 | 0.01-2 |
| 10 | Triethyl amine | Q.S. to adjust the pH |
| | | between about 5.0 and 6.5 |
| 11 | Purified water | Q.S. |

Q.S. = Quantity Sufficient

Method of Preparation:

The suspension dosage form of Cinacalcet was prepared according to the process mentioned below.
(a) Add methyl paraben and propyl paraben in purified water;
(b) Add sucralose, simethicone, polysorbate 80 and glycerin sequentially;
(c) Add Cinacalcet or pharmaceutically acceptable salt thereof;
(d) Add hydroxypropyl methyl cellulose, triethyl amine (to adjust the pH between about 5.0 and 6.5) and fraise flavor sequentially; and
(e) Add required quantity of purified water to make up the volume.

### Example-2: Stability Study Results of Liquid Dosage Form Prepared in Example-1

The liquid dosage form prepared according to the Example-1 was evaluated for their storage stability under different storage conditions. It was surprisingly found that the liquid dosage form of Cinacalcet is stable for prolonged time when tested under different storage conditions. The results of the stability studies conducted are summarized in the table below. These results also show that because of their prolonged storage stability, the liquid dosage forms of the present invention can become a useful alternative to the marketed drug (Sensipar®).

| | | Results | | | | | | |
|---|---|---|---|---|---|---|---|---|
| Test | | | 40° ± 2° C./25 ± 5 RH | | | | 25° ± 2° C./40 ± 5 RH | |
| Parameters | Specification | Initial | 1 M | 2 M | 3 M | 6 M | 3 M | 6 M |
| Description | White to | Complies | Complies | Complies | Complies | Complies | Complies | Complies |
| | brownish | | | | | | | |
| | white | | | | | | | |
| | suspension | | | | | | | |
| Assay of | 95-105% | 100.0% | 102.0% | 103.4% | 103.8% | 101.3% | 104.2% | 98.8% |
| Cinacalcet HCl | | | | | | | | |
| Assay of | 90-110% | 100.6% | 102.4% | 101.3% | 102.3% | 100.2% | 103.4% | 100.0% |
| methyl paraben | | | | | | | | |
| Assay of | 90-110% | 96.1% | 97.0% | 97.6% | 98.4% | 94.5% | 100.0% | 94.7% |
| propyl paraben | | | | | | | | |
| pH | Between about | 5.54 | 5.56 | 5.53 | 5.47 | 5.74 | 5.48 | 5.74 |
| | 5.5 and 6.5 | | | | | | | |

Related Substances

| | | | | | | | | |
|---|---|---|---|---|---|---|---|---|
| Amine impurity | NMT 0.2% | ND | 0.01% | 0.01% | 0.06% | ND | 0.02% | 0.01% |
| Alcohol impurity | NMT 0.2% | ND | ND | ND | ND | ND | ND | ND |
| Mesylated impurity | NMT 0.2% | ND | ND | ND | ND | ND | ND | ND |
| Regio impurity | NMT 0.2% | ND | ND | ND | ND | 0.02% | ND | ND |
| Dimer impurity | NMT 0.2% | 0.03% | 0.02% | 0.03% | ND | 0.01% | ND | ND |
| Single maximum | NMT 0.2% | 0.04% | 0.06% | 0.06% | 0.10% | 0.21% | 0.05% | 0.06% |
| impurity | | | | | | | | |
| Total impurities | NMT 2.0% | 0.15% | 0.23% | 0.23% | 0.38% | 0.23% | 0.14% | 0.14% |
| Viscosity | To be | 243.75 | 244.50 | 229.00 | 239.50 | 232.00 | 231.50 | 236.6 |
| | recorded | | | | | | | |

NMT = Not more than;
ND = Not delected

The liquid dosage forms of Cinacalcet prepared according to the present invention as described herein are suitable for use in the industry.

It should be understood that various changes and modifications to the embodiments described herein will be apparent to those skilled in the art. Such changes and modifications can be made without departing from the spirit and scope of the subject matter of the present invention and without diminishing its intended advantages. It is therefore intended that such changes and modifications be covered within the scope of the present invention.

We claim:

1. A liquid dosage form of Cinacalcet comprising Cinacalcet or pharmaceutically acceptable salt thereof and one or more pharmaceutically acceptable excipients or additives.

2. A liquid dosage form of Cinacalcet according to claim 1, wherein one or more pharmaceutically acceptable excipient or additive is selected from the group comprising of vehicles, solvents/co-solvents, solubilizers, solubility enhancing agents, suspending agents/thickening agents/viscosity modifying agents, permeation/penetration enhancers, tonicity agents, mucoadhesives, bulking agents/auxiliary suspending agents, chelating agents, wetting agents, anti-foaming agents, anti-caking agents, stabilizing agents, anti-oxidants, buffering agents and/or pH modifying agents and/or pH adjusting agents, surfactants, preservatives, sweetening agents, flavoring agents and coloring agents.

3. A liquid dosage form of Cinacalcet according to claim 1 or claim 2, wherein the liquid dosage form is selected from the group comprising of liquids, liquid dispersions, suspensions, solutions, emulsions, sprays, ointments, creams, spot-on, syrups, elixirs, drops, gels, solution-gels and concentrates.

4. A liquid dosage form of Cinacalcet according to any one of claims 1 to 3 is suitable for administration selected from the group comprising of oral, pulmonary, intravenous, rectal, colonic, parenteral, intracisternal, intraperitoneal, local, buccal, nasal and topical administration.

5. A liquid dosage form of Cinacalcet according to any one of claims 1 to 4, wherein the liquid dosage form is solution suitable for oral administration.

6. A liquid dosage form of Cinacalcet according to any one of claims 1 to 4, wherein the liquid dosage form is suspension suitable for oral administration.

7. A liquid dosage form in the form of solution suitable for oral administration, comprising Cinacalcet or pharmaceutically acceptable salt thereof, and one or more pharmaceutically acceptable excipients or additives selected from the group comprising of vehicles, solvents/co-solvents, solubilizers, preservatives, surfactants, pH adjusting agents and/or pH modifiers and/or buffering agents, sweetening agents, flavoring agents and coloring agents.

8. A liquid dosage form in the form of suspension suitable for oral administration, comprising Cinacalcet or pharmaceutically acceptable salt thereof, and one or more pharmaceutically acceptable excipients or additives selected from the group comprising of vehicles, solvents/co-solvents, solubilizers, suspending agents/thickening agents/viscosity modifying agents, anti-foaming agents, anti-caking agents, preservatives, surfactants, pH adjusting agents and/or pH modifying agents and/or buffering agents, sweetening agents, flavoring agents and coloring agents.

9. A liquid dosage form of Cinacalcet according to claim 6 or claim 8 comprises particles of Cinacalcet or pharmaceutically acceptable salt thereof, wherein the d₉₀ of the particles are between about 10 μm and about 200 μm.

10. A liquid dosage form of Cinacalcet according to any one of claims 5 to 9, wherein the liquid dosage form is ready to use dosage form or prepared by reconstituting dry powder in suitable diluent or media.

11. A liquid dosage form of Cinacalcet according to any one of claims 5 to 10, wherein the liquid dosage form is an immediate release dosage form or a modified release dosage form.

12. A liquid dosage form of Cinacalcet according to any one of claims 5 to 11, wherein the pH of the dosage form is between about 3.0 and 8.5.

13. A liquid dosage form of Cinacalcet according to any one of claims 5 to 12, wherein the dosage form is stable for prolonged time when stored under typical storage conditions and/or accelerated conditions characterized in that any individual impurity present in the liquid dosage form is less than 2.0% and the total impurities present in the liquid dosage form are less than 5.0%.

14. A liquid dosage form of Cinacalcet according to any one of claims 5 to 13, wherein the liquid dosage form has:
(a) a Cmax for Cinacalcet, or a salt or derivative thereof, when assayed in the plasma of a mammalian subject following administration that is at least about 50% to about 1900% greater than the Cmax for an Cinacalcet marketed or known formulation, administered at the same dose;
(b) an AUC for Cinacalcet, or a salt or derivative thereof, when assayed in the plasma of a mammalian subject following administration that is at least about 25% to about 1200% greater than the AUC for an Cinacalcet marketed or known formulation, administered at the same dose;
(c) a Tmax for Cinacalcet, or a salt or derivative thereof, when assayed in the plasma of a mammalian subject following administration that is less than about 6 hours to about 8 hours; or
(d) any combination of (a), (b), and (c).

15. A process for the preparation of the liquid dosage form according to any one of claim 5, claim 6, claim 7 or claim 8 comprising following steps:
(a) One or more preservatives are added in the vehicle;
(b) One or more sweetener, optionally one or more antifoaming agents, optionally one or more surfactants and one or more solvents/co-solvents or solubilizers are added in step (a);
(c) Cinacalcet or pharmaceutically acceptable salt thereof is added in step (b);
(d) Optionally one or more suspending agents/thickening agents/viscosity modifying agents, one or more pH adjusting agents and/or pH modifying agents and/or buffering agents and one or more flavoring agents and/or one or more solvents/co-solvents are added in step (c); and
(e) Vehicle is added to adjust the final volume.

16. Use of a liquid dosage form according to any one of claims 1 to 14 in the treatment of one or more diseases or conditions selected from the group comprising of hyperparathyroidism, such as primary hyperparathyroidism and secondary hyperparathyroidism, hyperphosphonia, hypercalcemia, elevated calcium-phosphorus product, osteoporosis, arterial stiffness, anemia, familial hypophosphatemic rickets, hemodialysis, parathyroid tumors, vascular diseases, recurrent prostate cancer, adenocarcinoma of prostate, parathyroid adenoma, parathyroid hyperplasia, renal osteodystrophy, osteomalacia, memory functions, familial primary hyperparathyroidism, parathyroid neoplasms, coronary artery calcification and cardiovascular diseases.

17. A liquid dosage form of Cinacalcet according to any one of claims 1 to 14 is packaged in the pharmaceutically acceptable packaging material selected from the group comprising of containers, pumps, bottles with spray pump, bottles with dropper assembly, bottles, collapsible tubes, glass ampoules, stoppered vials, pre-filled syringes, wherein the bottles or containers are clear/transparent/opaque or amber colored glass bottles and containers or clear/transparent/opaque or amber colored plastic bottles and containers made from polyethylene, low-density polyethylene, high-density polyethylene, polyamide, polyolefin, polycarbonate, acrylic multipolymers, polypropylene, polyethylene terephthalate, polyvinyl chloride, polystyrene.
